(19) United States
(12) Patent Application Publication
(10) Pub. No.: US 2025/0279168 A1
(43) Pub. Date: Sep. 4, 2025

(54) Integrated System for Predictive Recruitment and Direct Engagement in Veterinary Clinical Trials

(71) Applicant: Susie Nayoung Kim, Moss Beach, CA (US)

(72) Inventor: Susie Nayoung Kim, Moss Beach, CA (US)

(21) Appl. No.: 18/595,319

(22) Filed: Mar. 4, 2024

(51) Int. Cl. G06Q 10/1053

(52) U.S. Cl. CPC G16H 10/20

(57) ABSTRACT

Disclosed is an automated system and method for enhancing the recruitment process of clinical trial candidates within the veterinary field. It utilizes a data-driven approach to predict the recruitment value of veterinary clinics based on their historical purchase data and operational information. The system facilitates direct connections between animal health manufacturers and veterinary clinics, enabling manufacturers to efficiently recruit suitable clinical trial candidates and offer customizable rewards to participating clinics. This innovative platform streamlines the recruitment workflow, reduces dependency on intermediaries, and fosters direct financial transactions, thereby expediting the clinical trial process and creating new opportunities for veterinary clinics to engage in research collaborations.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO AN APPENDIX SUBMITTED ON A COMPACT DISC AND INCORPORATED BY REFERENCE OF THE MATERIAL ON THE COMPACT DISC

Not applicable.

### STATEMENT REGARDING PRIOR DISCLOSURES BY THE INVENTOR OR A JOINT INVENTOR

Reserved for a later date, if necessary.

### BACKGROUND OF THE INVENTION

### Field of Invention

The disclosed subject matter is in the field of integrated digital platforms for the veterinary sector, specifically designed to automate and optimize the recruitment process for clinical trial candidates by utilizing advanced data analytics and direct engagement mechanisms between veterinary clinics and manufacturers.

### Background of the Invention

Veterinary clinics and animal health and medicine manufacturers face significant challenges in clinical trial recruitment and purchasing management. Traditionally, veterinary clinics use manual and tedious purchasing and vendor management processes. This inefficient tradition consumes valuable time and limits the clinics' ability to engage in broader activities, such as participating in clinical trials or exploring opportunities as Key Opinion Leaders (KOLs) or in sponsored speakership roles. On the other side, animal health and medicine manufacturers traditionally struggle with recruiting clinical trial candidates due to the absence of a centralized database for veterinary clinical trials, akin to those available in human medical research. This gap forces such manufacturers to rely on intermediaries for recruitment, incurring excessive costs and experiencing delays in drug development due to the cumbersome recruitment process.

### SUMMARY OF THE INVENTION

In view of the foregoing, an object of this specification is to disclose an integrated platform that automates the recruitment of clinical trial candidates for veterinary clinics and manufacturers. Suitably, the platform centers on automating veterinary clinical trial candidate recruitment by predicting recruitment value based on historical purchase data and operational data from veterinary clinics, matching the manufacturers involved in clinical studies directly with veterinary clinics, and enabling direct payments to clinics. The platform leverages a novel approach by utilizing historical purchase data and operational data from veterinary clinics to predict a clinic's recruitment value. This predictive capability, powered by Al, enables direct matching of manufacturers with veterinary clinics that are likely to have the target candidates for their clinical trials. By facilitating direct recruitment and enabling manufacturers to compensate veterinary clinics directly, the invention eliminates the need for intermediaries, thereby reducing costs and streamlining the recruitment process. This system not only benefits manufacturers by expediting drug development but also provides veterinary clinics with an additional revenue stream and the opportunity to contribute to the advancement of veterinary medicine.

The genesis of this innovative platform was realization of the significant challenges faced by manufacturers in recruiting clinical trial candidates. This disclosed platform stands out from prior technologies by providing a purchasing automation service that generates a rich source of data, which is leveraged to predict the match between a clinic's clientele base and a manufacturer's clinical trial eligibility profile. This approach represents a significant advancement over existing solutions, which either focus on human matchmaking in social networking contexts, as seen in U.S. Pat. No. 10,565,276 B2, or do not utilize purchasing and operational data for recruitment purposes, as seen in US patent.

### DESCRIPTION OF RELATED ART

Prior art documents exist.

US20040093238A1 discloses a SYSTEM AND PROCESS FOR MATCHING PATIENTS WITH CLINICAL MEDICAL TRIALS but does not specify the use of purchasing data for recruitment.

U.S. Pat. No. 6,839,678 discloses a COMPUTERIZED SYSTEM FOR CONDUCTING MEDICAL STUDIES but the disclosed system neither targets the veterinary sector nor uses purchasing and operational data from veterinary clinics to predict recruitment value.

Additionally, this patent focuses on determining patient eligibility for medical studies based on predefined criteria and managing the study sequence instead of emphasizing, as disclosed here, the prediction of a clinic's recruitment value and direct engagement between manufacturers and clinics.

US20020099570A1 discloses RECRUITING A PATIENT INTO CLINICAL TRIAL and offers relevant background on patient recruitment systems. However, this document fails to disclose any direct application to veterinary clinics and does not disclose the use of purchasing data for recruitment purposes.

US20020002474A1 discloses SYSTEMS AND METHODS FOR SELECTING AND RECTUIRINT INVESTIGATOR AND SUBJECTS FOR CLINICAL TRIALS and is directed to an integrated online interactive forum that facilitates the exchange of information among clinical study sponsors, investigators, and potential subjects. The document fails to disclose automated veterinary clinical trial candidate recruitment by predicting recruitment value based on historical purchase data and operational data from veterinary clinics, matching manufacturers directly with veterinary clinics, and enabling direct payments to clinics.

US20050038692A1 discloses a SYSTEM AND METHOD FOR FACILITATING CENTRALIZED CANDIDATE SELECTION AND MONITORING SUBJECT PARTICIPATING IN CLINICAL TRIAL STUDIES and emphasizes the training of raters and the standardization of candidate assessment to improve inter-rater reliability and reduce bias in the clinical trial processes. This is a different approach than the disclosed data-driven predictive model geared toward a system that facilitates direct connections between manufacturers and veterinary clinics and includes a mechanism for direct payments.

### BRIEF DESCRIPTION OF THE OF THE DRAWINGS

Other objectives of the disclosure will become apparent to those skilled in the art once the invention has been shown and described. The way these objectives and other desirable characteristics are achieved is explained in the following description and attached figures in which:

FIG. 1 is an exemplary diagram of the disclosed system;

FIG. 2 is a table; and,

FIG. 3 is a flow chart of the clinic side and manufacturing side of the system.

It is to be noted, however, that the appended figures illustrate only typical embodiments of this invention and are therefore not to be considered limiting of its scope, for the invention may admit to other equally effective embodiments that will be appreciated by those skilled in the relevant arts. Also, figures are not to scale but are instead representative.

### DETAILED DESCRIPTION OF PREFERRED EMBODIMENTS

Disclosed is an automated system and method for enhancing the recruitment process of clinical trial candidates within the veterinary field. The system and method utilize a data-driven approach to predict the recruitment value of veterinary clinics based on their historical purchase data and operational information. The system enables direct connections between animal health and medicine manufacturers and veterinary clinics, facilitating manufacturers to efficiently recruit suitable clinical trial candidates and offer customizable rewards to participating clinics. This disclosed platform streamlines the recruitment workflow, reduces dependency on intermediaries, and fosters direct financial transactions, thereby expediting the clinical trial process and creating new opportunities for veterinary clinics to engage in research collaborations. The more specific details of this technology are disclosed with reference to the figures.

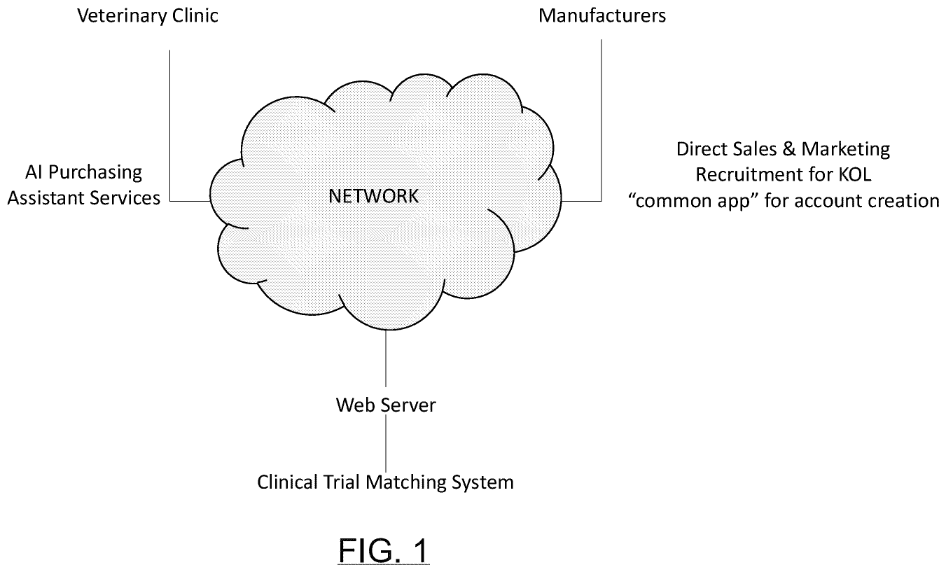

FIG. 1 is an exemplary diagram of the disclosed system. As illustrated, at least one veterinary clinic and at least one health or medicine manufacturer or vendor connect via a cloud-based network. Suitably, the veterinary clinic interacts with the manufacturer or vender over cloud-based network using an artificially intelligent purchasing assistant service such that medicines and other health and wellness products are purchased by the clinic from the manufacturer/vendor. On the other hand, the manufacturer/vendor interacts with the veterinary clinic including over the cloud-based network via direct sales and marketing or for recruitment for KOL. In other cases, the system involves a software application that is common to both the manufacturers/vendors and the clinics. Suitably, the software application runs on a webserver through the cloud-based network and includes a clinical trial matching system.

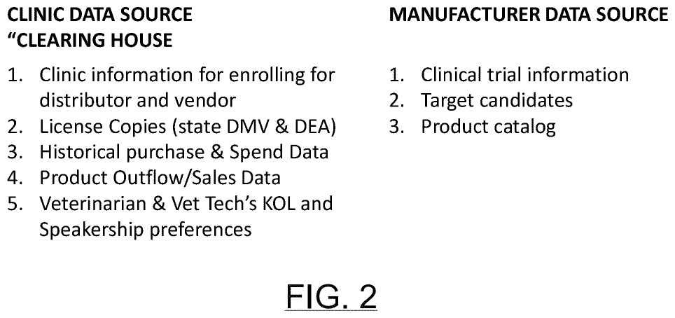

FIG. 2 is a table of clinical data sources and manufacturer/vendor data sources. As shown, clinical data sources include: 1. clinical information for enrolling for distributors and vendors; 2. License copies (of state DVM and DEA license); 3. Historical purchase and spend data; 4. Product outflow/sales data; and 5. Veterinarian and vet. Tech's KOL and speakership preferences. As shown, manufacturer data sources include: 1. Clinical trial information; 2. Target candidate information; 3. Product catalogs.

Suitably, the common software app discussed in connection with FIG. 1 features modules. Specifically, the software features:

Purchasing Module: This component automates the purchasing process for veterinary clinics, allowing them to efficiently manage orders and inventory while providing valuable data on purchasing patterns.

Clinical Trial Recruiting Module: This module uses the data from the Purchasing Module to match veterinary clinics with manufacturers seeking clinical trial candidates. It enables direct recruitment by manufacturers, bypassing the need for intermediaries.

Clinic Setting Module: This part of the system allows clinics to customize their settings and preferences, which can include their interest in participating in clinical trials or becoming Key Opinion Leaders (KOLs) or sponsored speakers.

Recruitment Value Predictability Module: Leveraging AI, this module predicts the recruitment value of a veterinary clinic based on their historical purchase data and clinic information, including their likelihood of having the target candidates for specific clinical trials.

Messaging Module: This facilitates communication between manufacturers and veterinary clinics or professionals who have expressed interest in KOL or ambassador roles.

Matching Module: It pairs veterinary clinics with manufacturers' clinical trial needs based on the predicted recruitment value and other relevant criteria.

Dashboard Module: This provides a user-friendly interface for both clinics and manufacturers to manage their interactions, track progress, and view analytics.

Implementation of the system includes software programed with the following objectives, functions, and data utilization:

### Purchasing Module

Objective: Automate the purchasing process for veterinary clinics.
Functionality: Manage orders and inventory, collect data on purchasing patterns.
Data Utilization: Use this data to inform other modules about clinic purchasing behaviors.

### Clinical Trial Recruiting Module

Objective: Match veterinary clinics with manufacturers seeking clinical trial candidates.
Functionality: Utilize data from the Purchasing Module to identify potential matches.
Process: Enable direct recruitment by manufacturers, bypassing intermediaries.

### Clinic Setting Module

Objective: Allow clinics to customize their settings and preferences.
Functionality: Include preferences for participating in clinical trials or roles like Key Opinion Leaders (KOLs) or sponsored speakers.

### Recruitment Value Predictability Module

Objective: Predict the recruitment value of a veterinary clinic.
Functionality: Leverage Al to analyze historical purchase data and clinic information.
Outcome: Determine clinics' likelihood of having the target candidates for specific clinical trials.

### Messaging Module

Objective: Facilitate communication between manufacturers and veterinary clinics.
Functionality: Support interactions for clinics interested in KOL or ambassador roles.

### Matching Module

Objective: Pair veterinary clinics with manufacturers' clinical trial needs.
Functionality: Use the Recruitment Value Predictability Module's output and other criteria for matching.

### Dashboard Module

Objective: Provide a user-friendly interface for clinics and manufacturers.
Functionality: Manage interactions, track progress, and view analytics.

### Implementation Considerations

Data Security and Privacy: Ensure the protection of sensitive data from both clinics and manufacturers.
Scalability: Design the system to manage growth in the number of users and data volume.
User Experience: Create intuitive interfaces for different user roles.
Integration: Allow for seamless integration with existing systems in veterinary clinics and manufacturers.
Compliance: Adhere to regulations governing veterinary practices and clinical trials.

Suitably, each module will involve a suitable logic flow as set forth below:

### Purchasing Module

### 1. User Authentication & Authorization

Step 1: Implement secure login mechanisms for veterinary clinics.
Step 2: Assign roles and permissions ensuring only authorized personnel can make purchases or view purchasing data.

### 2. Vendor and Product Catalog Management

Step 3: Create a database to store information about vendors, products, and prices.
Step 4: Develop a graphic user interface for clinics to browse, search, and select products from a comprehensive catalog.

### 3. Order Management

Step 5: Design a digital shopping cart where users can add or remove products.
Step 6: Implement order placement functionality with quantity selection and order confirmation.

### 4. Purchase Data Collection

Step 7: Automatically record and store detailed purchase data for each transaction, including product types, quantities, purchase dates, and costs.

### 5. Data Analysis and Reporting

Step 8: Develop algorithms configured to analyze purchasing patterns and to identify trends such as frequently purchased products or seasonal variations in purchasing behavior.
Step 9: Create report-style tools that allow clinics to review their purchasing history or other insights derived from data analysis.

### 6. Integration With Other Modules

Step 10: Ensure seamless data flow from the Purchasing Module to the Clinical Trial Recruiting Module and the Recruitment Value Predictability Module including via establishing APIs or data sharing mechanisms that permit other modules to access and utilize purchasing data.

### 7. User Interface and Experience

Step 11: Design an intuitive user interface for the Purchasing Module, ensuring easy navigation, product selection, and order management.
Step 12: Implement feedback mechanisms, such as order confirmation messages and alerts for low inventory or exclusive offers.

### 8. Security and Compliance

Step 13: Incorporate data encryption, secure data storage, and privacy measures to protect sensitive information.
Step 14: Ensure the system complies with relevant regulations and standards governing veterinary practices and data protection.

### 9. Testing and Iteration

Step 15: Conduct thorough testing of the Purchasing Module, including unit tests, integration tests, and user acceptance testing.
Step 16: Gather feedback from early users and iterate on the design and functionality to improve user experience and system performance.

### Clinical Trial Recruiting Module

### 1. Data Integration

Step 1: Integrate the module with the Purchasing Module to access historical purchase data of veterinary clinics.
Step 2: Integrate with the Clinic Setting Module to incorporate clinics' preferences for participating in clinical trials or roles such as KOLs.

### 2. Manufacturer Requirements Input

Step 3: Create an interface for manufacturers to input their clinical trial requirements, including the type of candidates needed, trial specifications, and any other relevant criteria.

### 3. Clinic Recruitment Value Analysis

Step 4: Utilize the Recruitment Value Predictability Module to analyze clinic data and predict which clinics have the highest potential for matching the trial requirements.

### 4. Matching Logic

Step 5: Develop an algorithm that matches clinics with manufacturers based on the recruitment value scores and the trial requirements specified by the manufacturers.

### 5. Direct Recruitment Facilitation

Step 6: Implement functionality for manufacturers to directly contact matched clinics, using the Messaging Module for communication.

### 6. Customizable Rewards System

Step 7: Design a system that allows manufacturers to offer customizable rewards or payments to clinics for successful recruitment.

### 7. Tracking and Feedback

Step 8: Integrate with the Dashboard Module to provide both clinics and manufacturers with the ability to track recruitment progress, view matches, and receive feedback.

### 8. Security and Compliance

Step 9: Ensure all data exchanges comply with privacy laws and industry regulations, implementing secure data handling and transfer protocols.

### 9. User Experience

Step 10: Create a user-friendly interface for both clinics and manufacturers to easily navigate through the recruitment process.

### 10. Testing and Iteration

Step 11: Conduct thorough testing, including unit, integration, and user acceptance tests, to ensure the module functions correctly and efficiently.
Step 12: Collect user feedback and make necessary adjustments to improve the module's performance and usability.

### Clinic Setting Module

### 1. User Authentication and Profile Setup

Step 1: Implement secure user authentication to ensure that only authorized clinic personnel can access and modify settings.
Step 2: Allow clinics to create and edit their profiles, including basic information and areas of specialization.

### 2. Preferences Configuration

Step 3: Design a user interface where clinics can specify their preferences for participating in clinical trials. This could include checkboxes or toggles for opting in or out of trial participation, interest areas, and preferred contact methods.
Step 4: Provide options for clinics to indicate their interest in KOL or sponsored speakership opportunities, including fields to describe their expertise and past experiences.

### 3. Data Integration

Step 5: Ensure the module can access and update the clinic's historical purchase data and operational data to enrich the clinic's profile for better matching with manufacturers.
Step 6: Integrate with the Recruitment Value Predictability Module to reflect changes in clinic preferences that might affect their recruitment value score.

### 4. Notification Settings

Step 7: Implement notification settings that allow clinics to customize how they receive alerts about matching clinical trial opportunities or KOL/speakership roles.

### 5. Privacy and Data Sharing Preferences

Step 8: Include privacy settings that let clinics control the visibility of their profile and data to manufacturers and other third parties.

### 6. Saving and Updating Preferences

Step 9: Provide a mechanism to save and update preferences in real-time, ensuring that any changes are immediately reflected across the platform.
Step 10: Validate input data for correctness and completeness before saving to prevent errors and inconsistencies.

### 7. User Interface and Experience

Step 11: Design an intuitive and user-friendly interface for the Clinic Setting Module, ensuring easy navigation and clear options for setting preferences.
Step 12: Offer help text or tooltips to guide users through setting up their preferences and understanding the implications of their choices.

### 8. Testing and Feedback

Step 13: Conduct thorough testing, including user acceptance testing, to ensure the module meets the needs of veterinary clinics and functions as intended.
Step 14: Collect feedback from early users to identify areas for improvement and refine the module accordingly.

### Recruitment Value Predictability Module

### 1. Data Collection and Integration

Step 1: Integrate with the Purchasing Module to access historical purchase data of veterinary clinics.
Step 2: Integrate with the Clinic Setting Module to incorporate clinics' preferences and additional operational data.

### 2. Feature Extraction

Step 3: Analyze the collected data to identify key features relevant to predicting a clinic's recruitment value. This could include types of products purchased, purchase frequency, clinic size, and expressed interest in clinical trials or KOL activities.

### 3. Data Preprocessing

Step 4: Clean and preprocess the data to ensure it is in a suitable format for analysis. This may involve handling missing values, normalizing data, and encoding categorical variables.

### 4. Model Selection

Step 5: Choose appropriate machine learning algorithms for predicting recruitment value. Consider regression models, decision trees, or ensemble methods like random forests or gradient boosting machines, depending on the data characteristics and prediction goals.

### 5. Training and Validation

Step 6: Split the data into training and validation sets. Train the selected models on the training set and validate their performance using the validation set. Employ cross-validation techniques to ensure robustness and avoid overfitting.

### 6. Model Optimization

Step 7: Tune the models' hyperparameters to optimize performance. Use techniques like grid search or random search to find the best parameter combinations.

### 7. Model Deployment

Step 8: Deploy the best-performing model into the production environment. Ensure the model can access real-time or regularly updated data for making predictions.

### 8. Prediction Interface

Step 9: Develop an API or interface that allows other modules, especially the Clinical Trial Recruiting Module, to query the Recruitment Value Predictability Module for clinic recruitment value scores.

### 9. Continuous Learning

Step 10: Implement mechanisms for the model to learn continuously from new data. This could involve periodic retraining or more sophisticated online learning approaches.

### 10. Monitoring and Evaluation

Step 11: Set up a system to monitor the model's performance over time, including tracking prediction accuracy and responding to any drift in data patterns.

### 11. User Feedback Loop

Step 12: Incorporate feedback mechanisms that allow users (veterinary clinics and manufacturers) to provide input on the recruitment value predictions, which should further refine and improve the model.

### Messaging Module

### 1. User Authentication and Role Verification

Step 1: Implement secure user authentication to ensure that only verified manufacturers and veterinary clinics or professionals can access the messaging features.
Step 2: Verify the roles and permissions of users to ensure manufacturers can only message clinics that have opted in for communication.
   2. Integration with Other Modules
Step 3: Integrate with the Clinic Setting Module to access clinics' preferences regarding receiving messages and their interest in KOL or ambassador roles.
Step 4: Integrate with the Matching Module to identify clinics and professionals that match manufacturers' criteria for clinical trial recruitment or KOL opportunities.

### 3. Messaging Interface

Step 5: Develop a user-friendly messaging interface that allows manufacturers to compose and send messages to selected clinics or professionals.
Step 6: Implement a system for veterinary clinics and professionals to receive, view, and respond to messages from manufacturers.

### 4. Communication Templates and Customization

Step 7: Provide manufacturers with customizable message templates for common communication needs, such as trial recruitment invitations or KOL opportunity proposals.
Step 8: Allow for personalization of messages to include specific details about the clinical trial or KOL opportunity.

### 5. Notification System

Step 9: Implement a notification system to alert clinics and professionals of new messages and manufacturers of received responses.
Step 10: Provide options for users to customize their notification preferences.

### 6. Privacy and Security

Step 11: Encrypt and securely store any messages.
Step 12: Prevent spam and unauthorized communication.

### 7. Message Tracking and History

Step 13: Provide tracking functionality for both manufacturers and clinics to track the status of sent and received messages.
Step 14: Log a history of communications for reference and compliance purposes.

### 8. Feedback and Reporting

Step 15: Accept feedback on the messaging experience and log reports of any issues or abuse.
Step 16: Generate reports on messaging activity and engagement metrics.

### 9. Testing and Iteration

Step 17: Test, including user acceptance tests, to ensure the Messaging Module intended functions.
Step 18: Collect user feedback and iterate accordingly on the module's design and functionality to enhance user experience and effectiveness.

### Matching Module

### 1. Data Integration

Step 1: Integrate the module with the Recruitment Value Predictability Module to receive recruitment value scores for clinics.
Step 2: Integrate with the Clinic Setting Module to access clinics' preferences and willingness to participate in clinical trials.

### 2. Manufacturer Criteria Input

Step 3: Develop an interface for manufacturers to input their criteria for clinical trial candidates, including specific medical conditions, treatment types, and demographic requirements.

### 3. Clinic Eligibility Assessment

Step 4: Implement logic to assess the eligibility of clinics based on manufacturers' input criteria and the clinics' recruitment value scores.

### 4. Matching Algorithm

Step 5: Design an algorithm that matches eligible clinics with manufacturers' needs, prioritizing clinics with higher recruitment value scores and alignment with the trial's requirements.

### 5. Match Presentation and Selection

Step 6: Present potential matches to manufacturers through a user-friendly interface, allowing them to review and select clinics for recruitment.

### 6. Direct Engagement Facilitation

Step 7: Use the Messaging Module to enable manufacturers to initiate contact with selected clinics to discuss trial participation and negotiate terms.

### 7. Match Tracking and Management

Step 8: Integrate with the Dashboard Module to allow both manufacturers and clinics to track the status of matches, communications, and agreements.

### 8. Feedback Loop

Step 9: Implement a feedback system where both parties can provide input on the matching process, which should refine the matching algorithm.

### 9. Security and Compliance

Step 10: Ensure all data handling within the module complies with privacy laws and industry regulations, implementing secure data exchange protocols.

### 10. Testing and Iteration

Step 11: Conduct thorough testing, including unit, integration, and user acceptance tests, to ensure the module functions correctly and efficiently.
Step 12: Collect user feedback and iterate on the module's design and functionality to enhance the matching process and user experience.

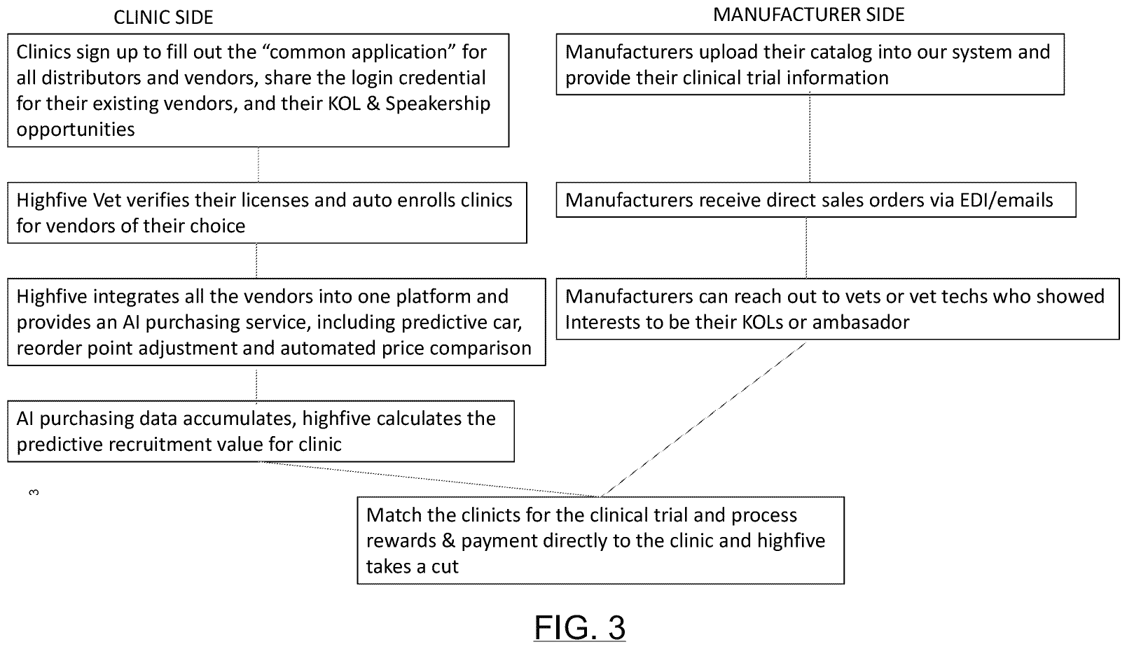

FIG. 3 is a flow chart of the system, in use. Referring to the chart, the clinic side involves the following steps: a. sign up to fill out the “common application” for all distributors vendors, and share log-in credentials for their existing vendors, and their KOL & Speakership opportunities preferences, and licenses; b. an administrator verifies the clinics licensing and enrolls clinics for the venders of their choice; c. the administrator integrates all the vendors into the platform and provides an artificially intelligent purchasing service, including predictive cart, reorder point adjustment and automated price comparison; d. as purchasing data accumulates, the administrator calculates the predictive recruitment value for the clinic. Referring to the chart, the manufacturing side involves the following steps: a. manufactures/vendors upload catalog into the system and provide clinical trial information; b. the manufacturers receive direct sales orders via EDI/emails; c. the manufacturers reach out to vets or vet techs who showed interest to their KOLs or ambassadors. Finally, the system matches the clinics for the clinical trial and processes rewards and payments directly to the clinic from the manufactures. Suitably, a fee for the matchmaking services provided to the system administrator.

An example of the system in action includes a veterinary clinic uses the platform to automate its purchasing process, which in turn feeds data into the system about what products they buy and how often. A manufacturer looking to conduct a clinical trial for a new canine arthritis drug can access the platform and use the Clinical Trial Recruiting Module to identify clinics that frequently purchase arthritis-related medications. The Recruitment Value Predictability Module analyzes this data to score and suggest clinics with a high likelihood of having eligible patients for the trial. The manufacturer can then use the Messaging Module to directly contact these clinics, propose a trial collaboration, and negotiate terms, including direct payment for their participation. The clinics benefit from this system by gaining an additional revenue stream and the opportunity to contribute to the advancement of veterinary medicine, while the manufacturer benefits from a more efficient and cost-effective recruitment process.

Another example includes a veterinary clinic uses the platform to automate its purchasing, feeding data into the system about their buying habits. A manufacturer seeking to conduct a clinical trial for a new drug uses the platform to identify clinics that frequently purchase related medications. The system's Recruitment Value Predictability Module scores clinics based on this and other data, suggesting potential matches. The manufacturer then directly contacts these clinics through the Messaging Module to propose collaboration.

In some embodiments, hardware and database elements are employed by the system, including:

### Server

Type: High-performance, multi-core server
Specifications: At least a 16-core processor (e.g., Intel Xeon or AMD EPYC) for efficient data processing and analytics. Minimum of 64 GB RAM to handle large datasets and simultaneous operations.
Purpose: To serve as the central processing unit for the system, executing complex algorithms for predictive analytics, managing the database operations, and handling user requests.

### Storage

Type: Enterprise-grade Solid State Drives (SSD) and Hard Disk Drives (HDD) Specifications: A combination of SSDs for fast access to frequently used data and larger capacity HDDs for long-term storage. A starting point could be 2 TB of SSD storage for the operating system, applications, and hot data, with 10 TB of HDD storage for archival and backup purposes.
Purpose: To store veterinary patient data, including medical records, diagnostic information, and trial eligibility criteria, ensuring fast retrieval times and secure, reliable long-term storage.

### Networking

Components: High-speed network interface cards (NICs), switches, and routers.
Specifications: Gigabit Ethernet or faster NICs in the server, with 10 GbE switches to support high-speed data transfers within the network.
Purpose: To ensure fast and secure communication between the server, storage components, and end-user devices. This setup supports the transfer of large datasets and enables real-time data access and updates.

### Software and Database Organization

Database Management System (DBMS): A robust DBMS like PostgreSQL or MongoDB, chosen based on the data structure and requirements (e.g., relational or NoSQL for unstructured data).
Software Modules:
   Data Ingestion Module: For importing and preprocessing incoming data from veterinary clinics and hospitals.
   Predictive Analytics Module: Utilizes machine learning algorithms to analyze patient data and predict trial eligibility.
   User Interface (UI) Module: Enables users to interact with the system, input data, and retrieve results.
Database Organization:
Schema Design: Carefully designed schema to optimize data storage, retrieval, and analysis. For relational databases, normalization to reduce redundancy; for NoSQL, a schema that supports the application's query patterns. Indexing: Implementing indexes on frequently queried fields to speed up data retrieval.
Security Measures: Encryption of sensitive data at rest and in transit, along with access controls to ensure data integrity and confidentiality.

Although the method and apparatus is described above in terms of various exemplary embodiments and implementations, it should be understood that the various features, aspects, and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus, the breadth and scope of the claimed invention are not limited by any of the above-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more,” or the like; and adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to,” or other like phrases in some instances shall not be read to mean that the narrower case is intended or required in instances where such broadening phrases might be absent. The use of the term “assembly” does not imply that the components or functionality described or claimed as part of the module are all configured in a common package. Indeed, any or all the various components of a module, whether control logic or other components, can be defined in a single package or separately maintained and might further be distributed across multiple locations.

Additionally, the various embodiments set forth herein are described in terms of exemplary block diagrams, flow charts, and other illustrations. As will become apparent to one of ordinary skill in the art after reading this document, the illustrated embodiments and their various alternatives might be implemented without confinement to the illustrated examples. For example, block diagrams and their accompanying description do not mandate a particular architecture or configuration.

This specification incorporates by reference all original claims in their entirety as if fully set forth herein.

I claim:

1. An automated system and method for enhancing the recruitment process of clinical trial candidates within the veterinary field.
